(19) United States
(12) Patent Application Publication
(10) Pub. No.: US 2025/0205965 A1
(43) Pub. Date: Jun. 26, 2025

(54) ORTHODONTIC ALIGNER FORMED OF THREE-DIMENSIONAL PRINTING COMPOSITION

(71) Applicant: GC Corporation, Shizuoka (JP)

(72) Inventors: Daisuke TAKADA, Tokyo (JP); Syuji KARIYA, Tokyo (JP)

(21) Appl. No.: 19/079,940

(22) Filed: Mar. 14, 2025

Related U.S. Application Data
(62) Division of application No. 17/260,034, filed on Jan. 13, 2021.

(30) Foreign Application Priority Data
Jul. 19, 2018 (JP) 2018-136108

(51) Int. Cl. B29C 64/106; B33Y 10/00; B33Y 70/00; B33Y 80/00; C07C 69/54; C08F 2/50; C08F 290/06

(52) U.S. Cl. CPC B29C 64/106; B33Y 10/00; B33Y 70/00; B33Y 80/00; C07C 69/54; C08F 2/50; C08F 290/067

(57) ABSTRACT

An orthodontic aligner that is formed of a three-dimensional printing composition includes: 10 to 80% by mass of (meth)acrylate having a urethane bond; 10 to 80% by mass of (meth)acrylate free from a urethane bond; 3 to 30% by mass of a polyurethane powder; and 0.01 to 10% by mass of a photopolymerization initiator. A viscosity of the composition is 200 to 3,000 mPa·s.

### CROSS-REFERENCE TO RELATED APPLICATIONS

The present application is a divisional application of and claims the benefit of priority under 35 U.S.C. 120 of U.S. patent application Ser. No. 17/260,034, filed on Jan. 13, 2021, which is a national stage application of PCT/JP2019/016834, which is based on and claims priority to Japanese Patent Application No. 2018-136108 filed Jul. 19, 2018, the entire contents of which are hereby incorporated by reference.

### FIELD OF THE INVENTION

The present invention relates to a three-dimensional printing composition and a method of producing a dental article.

### BACKGROUND OF THE INVENTION

In recent years, developments have been made in three-dimensional printing technologies. In the field of dentistry, denture bases, artificial teeth, and other dental articles have also been produced by three-dimensional printings (see, for example, Patent Document 1).

In contrast, clear resin aligners (mouthpieces) are used for orthodontics.

### RELATED-ART DOCUMENT

### Patent Documents

Patent Document 1: International Publication No. 2014/172716

### SUMMARY OF THE INVENTION

### Problems to be Solved by the Invention

However, there is a problem that when an orthodontic aligner is produced by three-dimensional printing, cracks are likely to generate in the orthodontic aligner during attachment and detachment.

One aspect is to provide a three-dimensional printing composition capable of suppressing cracking during attachment and detachment even when an orthodontic aligner is produced by three-dimensional printing.

### Means for Solving the Problems

One aspect is to provide a three-dimensional printing composition containing: 10 to 80% by mass of (meth)acrylate having a urethane bond; 10 to 80% by mass of (meth)acrylate free from a urethane bond; 3 to 30% by mass of a polyurethane powder; and 0.01 to 10% by mass of a photopolymerization initiator, wherein a viscosity of the composition is 200 to 3,000 mPa·s.

### Effects of the Invention

According to an aspect of the invention, a three-dimensional printing composition capable of suppressing cracking during attachment and detachment even when an orthodontic aligner is produced by three-dimensional printing.

### DETAILED DESCRIPTION OF THE INVENTION

Next, an embodiment for carrying out the present invention will be described.

### Three-Dimensional Printing Composition

A three-dimensional printing composition of the present embodiment contains (meth)acrylate having a urethane bond, (meth)acrylate free from a urethane bond, a polyurethane powder, and a photopolymerization initiator.

The (meth)acrylate refers to a compound (e. g., a monomer, oligomer, prepolymer, or the like) having a methacryloyloxy group and/or an acryloyloxy group (hereinafter referred to as (meth) acryloyloxy group).

The (meth)acrylate having a urethane bond preferably has two or more (meth) acryloyloxy groups, and particularly preferably has two (meth) acryloyloxy groups.

Examples of (meth)acrylates having a urethane bond include bis (2-(meth)acryloyloxyethyl)-2,2,4-trimethylhexamethylene dicarbamate; 1,3,5-tris[1,3-bis{(meth)acryloyloxy}-2-propoxycarbonylaminohexane]-1,3,5-(1H,3H,5H) triazine-2,4,6-trion; (meth)acrylate of urethane oligomers formed from 2,2′-bis(4-hydroxycyclohexyl)propane, 2-oxypanone, hexamethylene diisocyanate, and 2-hydroxyethylene (meth)acrylate; (meth)acrylate of urethane oligomers formed from 1,3-butanediol, hexamethylene diisocyanate, and 2-hydroxyethyl (meth)acrylate; and the like. Two or more kinds of (meth)acrylate having urethan bond may be used in combination.

A content of the (meth)acrylate having a urethane bond in a three-dimensional printing composition of the present embodiment is 10 to 80% by mass and preferably 25 to 75% by mass. When the content of (meth)acrylate having a urethane bond in the three-dimensional printing composition is less than 10% by mass, a polyurethane powder is easily precipitated, and a storage stability of the three-dimensional printing composition decreases. When the content of (meth)acrylate having a urethane bond in the three-dimensional printing composition is more than 80% by mass, a viscosity of the three-dimensional printing composition increases, and thus a dental article is not easily producible by three-dimensional printing.

A (meth)acrylate free from a urethane bond preferably has two or more (meth)acryloyloxy groups, and particularly preferably has two (meth)acryloyloxy groups.

Examples of (meth)acrylates free from a urethane bond include methyl (meth)acrylate, ethyl (meth)acrylate, isopropyl (meth)acrylate, n-butyl (meth)acrylate, isobutyl (meth)acrylate, hydroxypropyl (meth)acrylate, tetrahydrofurfuryl (meth)acrylate, glycidyl (meth)acrylate, 2-hydroxyethyl (meth)acrylate, 2-hydroxypropyl (meth)acrylate, 3-hydroxypropyl (meth)acrylate, 2-ethoxyethyl (meth)acrylate, 2-methoxyethyl (meth)acrylate, 2-ethylhexyl (meth)acrylate, benzyl (meth)acrylate, 2-hydroxy-1,3-di(meth)acryloxypropane, ethylene glycol di(meth)acrylate, diethylene glycol di(meth)acrylate, triethylene glycol di(meth)acrylate, butylene glycol di(meth)acrylate, neopentyl glycol di(meth)acrylate, 1,3-butanediol di(meth)acrylate, 1,4-butanediol di(meth)acrylate, 1,6-hexanediol di(meth)acrylate, trimethylol propane tri(meth)acrylate, trimethylol ethane tri(meth)acrylate, pentaerythritol tri(meth)acrylate, trimethylol methane tri(meth)acrylate, pentaerythritol tetra(meth)acrylate, polybutylene glycol di(meth)acrylate, ethoxylated bisphenol A di(meth)acrylate, bisphenol A diglycidyl (meth)acrylate, and the like. Two or more kinds may be used in combination.

A content of the (meth)acrylate free from a urethane bond in the three-dimensional printing composition of the present embodiment is 10 to 80% by mass and preferably 25 to 75% by mass. When the content of (meth)acrylate free from a urethane bond in the three-dimensional printing composition is less than 10% by mass, a viscosity of the three-dimensional printing composition increases, and thus a dental article is easily producible by three-dimensional printing. When the content of (meth)acrylate free from a urethane bond in the three-dimensional printing composition is more than 80% by mass, a polyurethane powder is easily to be precipitated, and a storage stability of the three-dimensional printing composition decreases.

A volume average particle size of the polyurethane powder is preferably 1 to 20 μm and more preferably 3 to 15 um. When the volume average particle size of the polyurethane powder is 1 μm or more, the viscosity of the three-dimensional printing composition of the present embodiment is further reduced, and thus a dental article is easily producible by three-dimensional printing. When the volume average particle size is 20 μm or less, the polyurethane powder is not easily precipitated, and a storage stability of the three-dimensional printing composition of the present embodiment further increases.

A content of the polyurethane powder in the three-dimensional printing composition of the present embodiment is 3 to 30% by mass, preferably 4 to 25% by mass, and more preferably 5 to 208 by mass. When the content of the polyurethane powder in the three-dimensional printing composition is less than 38 by mass, cracks are likely to generate in the orthodontic aligner during attachment and detachment even when an orthodontic aligner is produced by three-dimensional printing. When the content of the polyurethane powder in the three-dimensional printing composition is more than 30% by mass, a dental article is not easily producible by three-dimensional printing, because the viscosity of the three-dimensional printing composition increases.

Examples of photopolymerization initiators include camphorquinone, benzyl, diacetyl, benzyl dimethyl ketal, benzyl diethyl ketal, benzyl di(2-methoxyethyl)ketal, 4,4′-dimethyl (benzyl dimethyl ketal), anthraquinone, 1-chloroanthraquinone, 2-chloroanthraquinone, 1,2-benzanthraquinone, 1-hydroxyanthraquinone, 1-methylanthraquinone, 2-ethylanthraquinone, 1-bromoanthraquinone, thioxanthone, 2-isopropylthioxanthone, 2-nitrothioxanthone, 2-methylthioxanthone, 2,4-dimethylthioxantone, 2,4-diethylthioxanthone, 2,4-diisopropylthioxanthonton, 2-chloro-7-trifluoromethylthioxanthone, thioxanthone-10,10-dioxide, thioxanthone-10-oxide, benzoin methyl ether, benzoin ethyl ether, isopropyl ether, benzoin isobutyl ether, benzophenone, bis(4-dimethylaminophenyl)ketone, 4,4′-bis (diethylamino)benzophenone, (2,4,6-trimethylbenzoyl) diphenylphosphine oxide (TPO), bis(2,4,6-trimethylbenzoyl) phenylphosphine oxide, and the like. Two or more kinds may be used in combination.

A content of the photopolymerization initiator in the three-dimensional printing composition of the present embodiment is preferably 0.01 to 10% by mass and more preferably 0.1 to 58 by mass. When the content of the photopolymerization initiator in the three-dimensional printing composition is less than 0.01% by mass, and thus, the three-dimensional printing composition is not easily photo-cured. When the content of the photopolymerization initiator in the three-dimensional printing composition is more than 10% by mass, a dental article is more likely to become discolored even when the dental article is produced by three-dimensional printing.

The three-dimensional printing composition of the present embodiment may further contain fillers, pigments, UV absorbers, polymerization inhibitors, stabilizers, viscosity modifiers, and the like.

Forms of the three-dimensional printing composition of the present embodiment include liquids, pastes, and the like.

A viscosity of the three-dimensional printing composition of the present embodiment is 200 to 3,000 mPa·s and preferably 300 to 2,000 mPa·s. When the viscosity of the three-dimensional printing composition is less than 200 mPa·s or the viscosity thereof is more than 3,000 mPa·S, and thus, a dental article is not easily producible by three-dimensional printing.

Examples of dental articles include dental prostheses, orthodontic aligners, orthodontic retainers, surgical guides, splints (mouthpieces), indirect bonding trays, dental models, and the like. Of these, orthodontic aligners are preferred.

### Method of Producing Dental Articles

A method of producing a dental article according to the present embodiment includes a step of producing the dental article by three-dimensional printing using the three-dimensional printing composition of the present embodiment.

A publicly-known three-dimensional printer can be used in three-dimensional printing of dental articles using the three-dimensional printing composition of the present embodiment.

Examples of three-dimensional printing methods include a stereolithography (SLA) method, a digital light processing (DLP) method, and the like. Among these, the DLP method is preferably used.

A commercially available product of a DLP-type three-dimensional printer includes MAX385 (manufactured by ASIGA) and the like.

A method of three-dimensionally printing a dental article using the three-dimensional printing composition of the present embodiment includes, for example, a method of irradiating light from above a container containing a three-dimensional printing composition (a free liquid surface method) or a method of irradiating light from below a container (a regulated liquid level method). Among these, the regulated liquid level method is preferably applied.

When a dental article is produced by three-dimensional printing using the regulated liquid level method, the bottom surface of the container is light transmissive and light radiated from below the container passes through the bottom surface of the container and causing the three-dimensional printing composition to be irradiated with light.

Examples of light that the three-dimensional printing composition is irradiated with when a dental article is three-dimensionally produced by using a three-dimensional printing article of the present embodiment include, for example, ultraviolet light, visible light, or the like having a wavelength of 380 to 450 nm.

Examples of light sources of light radiated into the three-dimensional printing composition include LED lasers, LED lamps, LED projectors, and the like.

The method of producing a dental article of the present embodiment may further include a step of cleaning a three-dimensional printing article, a step of post-polymerizing a three-dimensional printing article, and the like.

The dental article produced by three-dimensional printing using the three-dimensional printing composition of the present embodiment preferably has a three-point bending strength of 10 to 80 MPa, an elastic modulus of 0.2 to 1.8 GPa, and a strain of 12 to 20%.

### EXAMPLE

Hereinafter, examples of the present invention will be described, but the present invention is not limited to the examples.

### Examples 1 to 6, Comparative Examples 1 to 4

A three-dimensional printing paste was prepared by mixing (meth)acrylate having a urethane bond with (meth)acrylate free from a urethane bond, a polyurethane powder, a photopolymerization initiator, and a polymerization inhibitor in a compounding amount (% by mass) indicated in Table 1.

The abbreviations in Table 1 are as follows. UDMA: Bis(2-methacryloyloxyethyl)-2,2,4-trimethylhexamethylene dicarbamate ((meth)acrylate having a urethane bond)

Biss MEPP 1: Ethoxylated bisphenol A dimethacrylate BPE-100 (manufactured by Shin-Nakamura Chemical Co., Ltd.) ((meth)acrylate free from a urethane bond)

Biss MEPP 2: Ethoxylated bisphenol A dimethacrylate BPE-500 (manufactured by Shin-Nakamura Chemical Co., Ltd.) ((meth)acrylate free from a urethane bond)

TEGDMA: Triethylene glycol dimethacrylate ((meth)acrylate free from a urethane bond)

TPO (2,4,6-trimethylbenzoyl) diphenylphosphine oxide (photopolymerization initiator)

BHT: Dibutyl hydroxytoluene (polymerization inhibitor)

Polyurethane powder (C-400): Urethane beads C-400 having a volume average particle diameter of 15 μm (manufactured by Negami Chemical Industrial Co., Ltd.)

Polyurethane powder (C-1000): Urethane beads C-1000 having a volume average particle diameter of 3 μm (manufactured by Negami Chemical Industrial Co., Ltd.)

### Viscosity

A viscosity of three-dimensional printing paste was measured using type B viscometer at 23° C.

### Preparation of Orthodontic Aligners

After a shape of an orthodontic aligner conforming to a tooth model was designed using the CAD Software Composer (manufactured by ASIGA), an orthodontic aligner was three-dimensionally produced by the DLP-typed three-dimensional printer MAX385 (manufactured by ASIGA) and the three-dimensional printing paste. Next, the three-dimensional printing article was thoroughly washed with isopropanol, and then post-polymerized using a dental photopolymerize device to prepare the orthodontic aligner.

### Testing for Cracks in Orthodontic Aligners

The orthodontic aligner was attached and detached from the tooth model and visually checked for cracks. The crack test was judged based on the following criteria.

Excellent: No cracks were observed.
Poor: Cracks were observed.

### Three-Point Bending Test of Test Piece

After a test piece of 2 mm×2 mm×25 mm was designed using the CAD Software Composer (manufactured by ASIGA), the test piece was three-dimensionally produced using the DLP-typed three-dimensional printer MAX385 (manufactured by ASIGA) and the three-dimensional printing paste. Next, the three-dimensional printing article was thoroughly washed with isopropanol, and then post-polymerized using a dental photopolymerizer to prepare the test piece.

The test piece was polished using water-resistant polishing paper and stored at 37° C. in water for 24 hours. Next, a three-point bending test of the test piece was performed at a crosshead speed of 1 mm/min using a small desktop tester EZ test (manufactured by Shimadzu Corporation), and the three-point bending strength was measured. In addition, an elastic modulus and a strain of the test piece were calculated from the results of the three-point bending strength.

Table 1 indicates the results of the aligner crack test and the three-point bend test of the test piece.

TABLE 1

| | Examples | | | | | | Comparative Examples | | | |
|---|---|---|---|---|---|---|---|---|---|---|
| | 1 | 2 | 3 | 4 | 5 | 6 | 1 | 2 | 3 | 4 |
| UDMA | 49.5 | 38.1 | 41.3 | 45.0 | 47.1 | 19.7 | 59.3 | 49.5 | 48.3 | 34.2 |
| B is M EPP 1 | 20.6 | 11.4 | 12.4 | 13.5 | 14.1 | 59.1 | 24.7 | 14.8 | 14.5 | 10.3 |
| B is M EPP 2 | | 11.4 | 12.4 | 13.5 | 14.1 | | | 14.8 | 14.5 | 10.3 |
| TEGDMA | 12.4 | 15.3 | 16.5 | 18.0 | 18.9 | 19.7 | 14.8 | 19.8 | 19.3 | 13.7 |
| TPO | 0.8 | 0.8 | 0.8 | 0.9 | 0.9 | 1.5 | 1.0 | 1.0 | 1.0 | 0.7 |
| BHT | 0.1 | 0.1 | 0.1 | 0.1 | 0.1 | 0.1 | 0.1 | 0.1 | 0.1 | 0.1 |
| Polyurethane powder | 16.5 | 22.9 | 16.5 | 9.0 | 4.7 | | | | 2.4 | 30.8 |
| (C-400) | | | | | | | | | | |
| Polyurethane powder | | | | | | 19.7 | | | | |
| (C-1000) | | | | | | | | | | |
| Total | 100 | 100 | 100 | 100 | 100 | 100 | 100 | 100 | 100 | 100 |
| Viscosity of paste [mPa · s] | 1700 | 1810 | 1060 | 640 | 480 | 680 | 670 | 380 | 410 | 3550 |
| Aligner crack test | Excellent | Excellent | Excellent | Excellent | Excellent | Excellent | Poor | Poor | Poor | — |

Bending test of test piece

| | | | | | | | | | | |
|---|---|---|---|---|---|---|---|---|---|---|
| Three-point bending | 47.6 | 47.0 | 60.9 | 70.4 | 77.2 | 19.8 | 80.8 | 88.0 | 81.6 | — |
| strength [MPa] | | | | | | | | | | |
| Elastic modulus [GPa] | 0.9 | 0.7 | 1.2 | 1.5 | 1.8 | 0.2 | 1.9 | 2.1 | 1.9 | — |
| Strain [%] | 18.8 | 18.5 | 15.1 | 12.6 | 12.8 | 18.0 | 13.5 | 8.0 | 10.1 | — |

From Table 1, it can be seen that when the orthodontic aligner was three-dimensionally produced using the three-dimensional printing paste of Examples 1 to 6, the generation of cracks during attachment and detachment can be suppressed.

In contrast, when the orthodontic aligner was three-dimensionally produced, cracks were generated during attachment and detachment because the three-dimensional printing paste of Comparative Examples 1 and 2 did not contain the polyurethane powder.

When the orthodontic aligner was three-dimensionally produced, cracks were generated during attachment and detachment, because the three-dimensional printing paste of Comparative Example 3 contained 2.48 by mass of polyurethane powder.

An orthodontic aligner was not able to be produced by the three-dimensional printing because the viscosity of the three-dimensional printing paste in Comparative Example 4 was 3, 550 mPa·s.

This international application is based on and claims priority of Japanese Patent Application No. 2018-136108 filed Jul. 19, 2018, the entire contents of which are hereby incorporated by reference.

1. An orthodontic aligner that is formed of a three-dimensional printing composition comprising:
10 to 80% by mass of (meth)acrylate having a urethane bond;
10 to 80% by mass of (meth)acrylate free from a urethane bond;
3 to 30% by mass of a polyurethane powder; and
0.01 to 10% by mass of a photopolymerization initiator, wherein a viscosity of the composition is 200 to 3,000 mPa·s.

2. An orthodontic aligner that is formed of a three-dimensional printing composition comprising:
10 to 80% by mass of (meth)acrylate having a urethane bond;
25 to 47.1% by mass of (meth)acrylate free from a urethane bond;
3 to 30% by mass of a polyurethane powder; and
0.01 to 10% by mass of a photopolymerization initiator,
wherein a viscosity of the composition is 200 to 3,000 mPa's that is measured by a type B viscometer at 23° C., pg,17
wherein the three-dimensional printing composition has an elastic modulus of 0.2 to 1.8 GPa and a strain of 12 to 20%.

3. The orthodontic aligner according to claim 2, wherein the (meth)acrylate free from the urethane bond includes at least two kinds different of ethoxylated bisphenol A dimethacrylate.
